(19) United States
(12) Patent Application Publication
(10) Pub. No.: US 2007/0134326 A1
(43) Pub. Date: Jun. 14, 2007

(54) Composition for releasing a weak base for an extended period of time

(76) Inventors: John Francis Hoke, Durham, NC (US); Luigi Martini, Harlow (GB); Vincenzo Re, Harlow (GB); Mark Edward Sale, Durham, NC (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/567,146

(22) PCT Filed: Aug. 6, 2004
(86) PCT No.: PCT/EP04/08843

§ 371 Date: Jul. 18, 2006

Related U.S. Application Data
(60) Provisional application No. 60/493,388, filed on Aug. 7, 2003.

(51) Int. Cl. A61K 9/22; A61K 31/4439

(52) U.S. Cl. 424/468; 514/338

(57) ABSTRACT

An oral dosage form comprising a first composition and a second composition, each composition comprising a pharmaceutically acceptable weak base, especially Compound A or a pharmaceutically acceptable salt or solvate thereof, (‘the drug’) and a pharmaceutically acceptable carrier therefor, wherein the first and second compositions are arranged to release drug at differing release rates on administration such that the rate of release of the drug from the dosage form is substantially independent of pH; a process for preparing such a dosage form and the use of such a dosage form in medicine.

The present invention relates to an oral dosage form comprising a pharmaceutically acceptable weak base, especially 5-[4-[2-(N-methyl-N-(2pyridyl)amino)ethoxy]benzyl]thiazolidine-2,4-dione (hereinafter ‘Compound A’) or a pharmaceutically acceptable salt or solvate thereof, to a process for preparing such a dosage form and to the use of such a dosage form in medicine.

The use of a coating to control the rate of release of an active agent has received considerable attention and many different devices have been developed for such a purpose. For example, International Patent Application, Publication Number WO 01/05430 describes a drug delivery device that enables the delivery of drug substances which exhibit pH dependent solubility, in particular compounds that are more soluble at low pH levels (less than pH 2) than at near neutral levels (greater than about pH 5). Such delivery devices are characterised by the presence of a coating that is impermeable and insoluble in the fluid of the environment of use.

International patent application, Publication Number WO 95/30422 describes a series of controlled-release dosage forms of azithromycin. In particular, there is described a series of dosage forms that reduce the exposure of the upper GI tract (e.g. the stomach) to high concentrations of azithromycin, by the use of a pH dependent coating. Such dosage forms do not feature openings through which release of the drug substance may occur.

U.S. Pat. No. 6,099,859 describes a controlled release tablet for the delivery of an antihyperglycaemic drug, which comprises an osmotically active drug-containing core and a semipermeable membrane, wherein the semipermeable membrane is permeable to the passage of water and biological fluids and is impermeable to the passage of the drug substance. The semipermeable membrane contains at least one passageway for the release of the antihyperglycaemic drug.

U.S. Pat. No. 5,543,155 describes a diffusion-osmotic controlled drug release pharmaceutical composition comprising a one- or two-layer tablet core containing hydroxypropyl methylcellulose, said core having a film-coat comprising an ammonium methacrylate copolymer.

Additional devices that utilise a coating to control the rate of release of an active agent are discussed in U.S. Pat. No. 5,004,614. This patent describes a tablet core provided with an outer coating that is substantially impermeable to environmental fluid. The said outer coating may be prepared from materials that are either insoluble or soluble in the environmental fluids. Where a soluble material is used, the coating is of sufficient thickness that the core is not exposed to environmental fluid before the desired duration of the controlled release of the active agent has passed. Through this impermeable outer coating, one or more opening(s) has been created, so as to provide environmental fluids with an access route to the core. Therefore, upon ingestion of the coated tablet, gastrointestinal fluid can enter the opening(s) and contact or penetrate the core, to release the active agent. The result is that the active agent is released in a controlled manner out of the opening(s) only. The preferred geometry is such that there is a circular hole on the top and bottom face of the coated tablet. The opening(s) in question have an area from about 10 to 60 percent of the face area of the coated tablet. The rate of drug release is found to be directly related to the diameter of the opening(s) and to the solubility of the matrix core and active agent, allowing the possibility for a variety of drug release profiles be it zero or first order release.

The substantially impermeable coatings of U.S. Pat. No. 5,004,614 are not suitable for the controlled release of all active agents, especially pharmaceutically active weak bases or pharmaceutically acceptable salts and solvates thereof. Such active agents exhibit a marked pH dependent solubility, i.e. they are more soluble at around pH 2, associated with regions found in the stomach, compared to their solubility in the generally neutral conditions of the small intestine, around pH 7.

International Patent Application, Publication Number WO 03/068195 discloses an oral dosage form comprising an erodable core which contains a pharmaceutically active weak base or a pharmaceutically acceptable salt or solvate thereof, such as Compound A, the core having a coating with one or more openings leading to the core, and the coating being erodable under predetermined pH conditions. This provides a beneficial means for administration of a pharmaceutically active weak base or a pharmaceutically acceptable salt or solvate thereof, such as Compound A, where it is desirable that release of the active compound takes place in more than one pH environment, based on the finding that it is also beneficial for the coating to be erodable or soluble in a pH dependent manner.

European Patent Application, Publication Number 0 306 228 A1 relates to certain thiazolidinedione derivatives disclosed as having antihyperglycaemic and hypolipidaemic activity. One particular thiazolidinedione disclosed in EP 0 306 228 A1 is Compound A. International Patent Application, Publication Number WO 94/05659 discloses certain salts of Compound A including the maleate salt at Example 1 thereof. Compound A or a pharmaceutically acceptable salt thereof or a pharmaceutically acceptable solvate thereof, may be prepared using known methods, for example those disclosed in EP 0 306 228 and WO 94/05659. The disclosures of EP 0 306 228 and WO 94/05659 are incorporated herein by reference.

Compound A and pharmaceutically acceptable salts or solvates thereof have useful pharmaceutical properties. In particular, Compound A or a salt or solvate thereof is indicated to be useful for the treatment and/or prophylaxis of diabetes mellitus, conditions associated with diabetes mellitus and certain complications thereof; Alzheimer's Disease, psoriasis, asthma, atherosclerosis, metabolic syndrome, impaired glucose tolerance and impaired fasting glucose.

International Patent Application, Publication Number WO 00/28990 describes various modified release pharmaceutical compositions comprising insulin sensitisers, including Compound A and pharmaceutically acceptable salts or solvates thereof.

International Patent Application, Publication Number WO 00/28990 describes a method of treating Type 2 diabetes mellitus and conditions associated with diabetes mellitus, using certain pharmaceutical compositions, including modified release compositions, which provide a Threshold Plasma Concentration of Compound A or a pharmaceutically acceptable salt or solvate thereof.

Compound A is a pharmaceutically acceptable weak base.

Compound A and pharmaceutically acceptable salts or solvates thereof, in particular the maleate salt, have been found to exhibit marked pH dependent solubility, i.e. they are more soluble in the acidic conditions of the stomach (around pH 2) than in the near neutral conditions of the lower intestine (around pH 7).

It is an object of the present invention to provide an oral dosage form comprising a pharmaceutically acceptable weak base, especially Compound A or a pharmaceutically acceptable salt or solvate thereof, which provides a maximised beneficial effect on glycaemic control for an extended period of time. Such a dosage form is considered to be suitable for once daily administration. Such a dosage form is also indicated for administration in both fasted and fed states, with substantially no clinically relevant food effect.

Accordingly, the present invention provides an oral dosage form comprising a first composition and a second composition, each composition comprising a pharmaceutically acceptable weak base, especially Compound A or a pharmaceutically acceptable salt or solvate thereof, (‘the drug’) and a pharmaceutically acceptable carrier therefor, wherein the first and second compositions are arranged to release drug at differing release rates on administration such that the rate of release of the drug from the dosage form is substantially independent of pH.

Suitably, the release rate of the drug from the first composition is substantially greater than from the second composition. It is envisaged that, the first composition is an immediate release composition. It is also envisaged that, the second composition is a modified release composition.

Suitably, the rate of release of the first and/or second composition(s) from the dosage form is a modified release. Preferably, said modified release is effected by a third composition, which third composition typically comprises substantially no drug substance. Said third composition is suitably an enteric composition, preferably a coating enteric layer, most preferably a non-permeable enteric coating layer, covering substantially all of the outer surface of the dosage form. In a preferred form said third composition comprises one or more openings extending substantially completely through the third composition, thereby exposing at least one surface of the first and/or second composition(s) to the environment of use.

In one aspect, the first composition is arranged so that in use it releases substantially all of the pharmaceutically acceptable weak base, such as Compound A or a pharmaceutically acceptable salt or solvate thereof, in the stomach.

In a further aspect, the second composition is arranged so that in use it releases substantially all of the pharmaceutically acceptable weak base, such as Compound A or a pharmaceutically acceptable salt or solvate thereof in the small intestine.

Suitably, the dosage form is a tablet form.

During human trials of an embodiment of the oral dosage form of the invention we have found that, release of the drug is such that the mean maximum plasma level concentration (“Cₘₐₓ”) value of the drug is maintained substantially independent of food during use, i.e. the observed Cₘₐₓ value is substantially similar in both fasted and fed states during use. Accordingly, in one aspect the the oral dosage form is arranged to release the pharmaceutically acceptable weak base, for example Compound A or a pharmaceutically acceptable salt or solvate thereof, such that the mean maximum plasma level concentration (“Cₘₐₓ”) value of the drug is maintained substantially independent of food during use, i.e. the observed Cₘₐₓ value is substantially similar in both fasted and fed states during use.

In addition it has also been found that the oral dosage form releases the drug such that the mean area under the plasma concentration versus time curve over the dosing interval at steady state (“AUC”) observed on administration is maintained substantially independent of food during use, i.e. the observed AUC is substantially similar in both fasted and fed states during use. Accordingly in one aspect the the oral dosage form is arranged to release the pharmaceutically acceptable weak base, for example Compound A or a pharmaceutically acceptable salt or solvate thereof, such that the mean area under the plasma concentration versus time curve over the dosing interval at steady state (“AUC”) is maintained substantially independent of food during use, i.e. the observed AUC is substantially similar in both fasted and fed states during use.

Thus, in a preferred aspect in operation the oral dosage form releases the pharmaceutically acceptable weak base, for example Compound A or a pharmaceutically acceptable salt or solvate thereof, so that both the Cₘₐₓ value and AUC observed on administration are maintained substantially independent of food during use, i.e. the observed Cₘₐₓ value and AUC are substantially similar in both fasted and fed states during use.

As is indicated herein, Compound A is a pharmaceutically acceptable weak base. It is anticipated that the dosage form of the present invention may be used to administer other pharmaceutically acceptable weak bases having similar physicochemical properties to Compound A, such as other weak bases.

As used herein the term “weak base” shall mean any base the conjugate acid of which has a pKa of less than 11.5; in accordance with The Pharmaceutical Handbook, 19th Edition, 1980, page 232. The term “pharmaceutically acceptable weak base” shall be interpreted accordingly. Suitable pharmaceutically acceptable weak bases or pharmaceutically acceptable salts or solvates thereof for use in the present invention include those compounds that exhibit a marked pH dependent solubility. Preferred pharmaceutically acceptable weak bases or pharmaceutically acceptable salts or solvates thereof for use in the present invention are more soluble in the pH range from 1 to 3 than in the pH range from 4.5 to 8, i.e they are more soluble in the acidic conditions found in the mammalian stomach than in the near neutral conditions of the mammalian intestines.

In a preferred embodiment the present invention provides an oral dosage form comprising,

(i) an erodable core, which core comprises a pharmaceutically acceptable weak base, especially Compound A or a pharmaceutically acceptable salt or solvate thereof, and a pharmaceutically acceptable carrier therefor; and

(ii) an erodable coating around said core, which coating comprises one or more openings extending substantially completely through said coating but not penetrating said core and communicating from the environment of use to said core, wherein release of pharmaceutically acceptable weak base, especially Compound A or a pharmaceutically acceptable salt or solvate thereof, from the erodable core occurs substantially through the said opening(s) and through erosion of said erodable coating under pre-determined pH conditions; wherein the core comprises a first composition and a second composition, each composition comprising a pharmaceutically acceptable weak base, especially Compound A or a pharmaceutically acceptable salt or solvate thereof, (‘the drug’) and a pharmaceutically acceptable carrier therefor, wherein the first and second compositions are arranged to release drug at differing release rates on administration such that the rate of release of the drug from the dosage form is substantially independent of pH.

Suitably, the first composition is formulated so that it provides immediate release of the pharmaceutically acceptable weak base, for example Compound A or a pharmaceutically acceptable salt or solvate thereof, on contact with aqueous media. Suitably, the second composition is formulated so that it provides modified release of a pharmaceutically acceptable weak base, for example Compound A or a pharmaceutically acceptable salt or solvate thereof, on contact with aqueous media.

The compositions can be formed in any shape or mutual conformation providing the required objective of the invention is met but generally each composition comprises a single layer of drug.

The above reference to the core being erodable includes the situation where the core disintegrates partially or wholly, or dissolves, or becomes porous, on contact with the relevant environmental fluid so as to allow the fluid to contact the active agent. Suitably, the core disintegrates partially. Suitably, the core disintegrates wholly. Suitably, the core dissolves. Suitably, the core becomes porous.

Whilst the preferred embodiment of this invention provides that erosion of the coating is pH dependent, the core may release the pharmaceutically acceptable weak base, such as Compound A or a pharmaceutically acceptable salt or solvate thereof, by eroding in a non-pH dependent manner.

Most suitably, although the pharmaceutically acceptable weak base, such as Compound A or a pharmaceutically acceptable salt or solvate thereof is more soluble in the stomach than the intestines, the core is formulated so as to release drug to substantially the same extent in both the stomach and the intestines, i.e. the core is formulated to compensate for the pH dependency of the pharmaceutically acceptable weak base, for example Compound A.

The above reference to the coating being erodable includes the situation where the coating disintegrates partially or wholly, or dissolves, or becomes porous, on contact with an environmental fluid so as to allow the fluid to contact the core. Suitably, the coating disintegrates partially. Suitably, the coating disintegrates wholly. Suitably, the coating dissolves. Suitably, the coating becomes porous. Preferably, the erodable coating is an enteric coating, i.e. it has a defined, pre-determined pH threshold at which it dissolves. Preferably, the coating erodes at pH greater than 4.5. More preferably, the coating erodes in the pH range from 4.5 to 8. Most preferably, the coating erodes in the pH rang 5 to 7. Preferably, the enteric coating is non-permeable.

Materials and their blends suitable for use as a pH-dependent erodable coating material in this invention include various polymethacrylate polymers, co-processed polyvinylacetate phthalate, cellulose acetate trimellitate, cellulose acetate phthalate, shellac, hydroxyropylmethylcellulose phthalate polymers and their copolymers. Suitably, the coating material is selected from cellulose acetate trimellitate (CAT), polyvinyl acetate phthalate, hydroxypropylmethylcellulose phthalate 50, hydroxpropyinethylcellulose phthalate 55, Acryl-eze™, Aquateric™, cellulose acetate phthalate, Eudragit™ L30 D, Eudragit™ L, Eudragit™ S and shellac. Most preferably, the coating material is Eudragit™ L30 D.

When necessary, the erodable coating may be modified by addition of plasticisers or anti-tack agents. Suitable materials for this purpose include waxy materials such as glycerides, for example glyceryl monostearate.

Typical sizes for the opening(s), when circular, to be formed in the coating are in the range 0.5 mm-8 mm of diameter, such as 1, 2, 3 or 4 mms in diameter, depending on the overall size of the tablet and the desired rate of release. The opening(s) may have any convenient geometrical shape, but a rounded shape, e.g. substantially circular or elliptical, is generally preferred. More elaborate shapes, such as text characters or graphics, may also be formed, provided that the release rate can be made uniform in individual dosage forms. Typical sizes of non-circular openings are equivalent in area to the above mentioned sizes for circular openings, thus in the range of from about 0.19 to about 50.3 mm².

For the purposes of the present invention, the term “opening” is synonymous with hole, aperture, orifice, passageway, outlet etc. The opening(s) may be formed by methods disclosed in U.S. Pat. No. 5,004,614. Typically opening(s) may be formed by drilling, for example using mechanical drill bits or laser beams, or by punches that remove the cut area. The formation of the opening(s) may by default remove a small portion of the exposed core. It is also possible to purposely form a cavity below the aperture as a release rate controlling device, the cavity exposing a greater initial surface area of core than a flat surface. Suitably, the opening(s) extend through the entire erodable coating such that there is immediate exposure of the core to the environmental fluid when the device is placed in the desired environment of use.

Also it is possible to form the opening(s) in situ when the dosage form is administered, by forming a coating containing pore-forming agents i.e. material that will dissolve in the stomach to create pores in the coating. Typically the pore forming agent is erodable in the pH range from 1 to 3.

In U.S. Pat. No. 5,004,614, the opening(s) preferably comprise about 10-60% of the total face area of the tablet i.e. the upper and lower surfaces of a biconvex tablet. In the present invention, the opening(s) may comprise 0.25 to 70%, such as 10-70% of the total face area.

Alternatively, it may be useful to characterise the rate controlling effect of the opening(s) by reference to the area of the opening(s) relative to the total surface area of the coated tablet. Additionally, especially in cases where the core erodes by undercutting of the edges of the opening(s), the rate controlling effect may be related to the total circumference of the opening(s).

A particularly unexpected finding is that two openings, for example one on each primary surface of a biconvex tablet, release an active agent from the core at a rate marginally greater than that of a single opening of the same overall area. It is also indicated that the variability of the release rate from the two openings is less than the variability of release rate from the corresponding single opening. Accordingly, in one embodiment of the invention, the coating of the core is provided with two or more openings. More preferably, the erodable coating surrounding the core is provided with two or more openings extending substantially completely through said coating but not penetrating said core and communicating from the environment of use to said core.

Where more than one opening is provided, the openings may be located on the same surface of the oral dosage form, or on different surfaces. Suitably, the oral dosage form has two openings, for example one on each of opposing surfaces. Suitably, the oral dosage form is a tablet having two opposed primary surfaces, each surface having one opening through the coating, preferably substantially completely through the coating. The core is suitably arranged so that one opening provides access to the first composition and the or another opening provides access to the second composition.

As a protection for the core material, to prevent contamination via the opening(s) before dosing, it may desirable to provide a conventional seal coating to either the core, or to the dosage form after formation of the opening(s). The seal coat may be a sub-coat or over-coat to the erodable coating.

According to yet a further aspect of the present invention, there is provided a process for preparing an oral dosage form which dosage form comprises a first composition and a second composition, each composition comprising a pharmaceutically acceptable weak base, such as Compound A or a pharmaceutically acceptable salt or solvate thereof, (‘the drug’) and a pharmaceutically acceptable carrier therefor, wherein the first and second compositions are arranged to release drug at differing release rates on administration such that the rate of release of the drug from the dosage form is substantially independent of pH;

which process comprises at least the steps of sequentially or simultaneously:

(i) formulating the drug into the first composition; and
(ii) formulating the drug into the second composition;
   whereby the first and second compositions are formulated to release drug at differing release rates on administration such that the rate of release of the drug from the dosage form is substantially independent of pH.

In a preferred aspect, there is provided a process for the preparation of an oral dosage form comprising (i) an erodable core, which core comprises a pharmaceutically acceptable weak base, such as Compound A or a pharmaceutically acceptable salt or solvate thereof and a pharmaceutically acceptable carrier therefor; and (ii) an erodable coating around said core, which coating comprises one or more openings extending substantially completely through said coating but not penetrating said core and communicating from the environment of use to said core, wherein release of a pharmaceutically acceptable weak base, such as Compound A or a pharmaceutically acceptable salt or solvate thereof from the erodable core occurs substantially through the said opening(s) and through erosion of said erodable coating under pre-determined pH conditions; characterised in that the core comprises a first composition and a second composition, each composition comprising the pharmaceutically acceptable weak base, such as Compound A or a pharmaceutically acceptable salt or solvate thereof (‘the drug’) and a pharmaceutically acceptable carrier therefor, wherein the first and second compositions are arranged to release drug at differing release rates on administration such that the rate of release of the drug from the dosage form is substantially independent of pH; which process comprises:
(a) formulating an erodable core comprising the pharmaceutically acceptable weak base, such as Compound A or a pharmaceutically acceptable salt or solvate thereof and a pharmaceutically acceptable carrier therefor;
(b) coating the said core with an erodable coating; and
(c) creating one or more openings in the coating, said openings extending substantially completely through said coating but not penetrating said core and communicating from the environment of use to said core.

The first and second compositions may be prepared by compressing suitable ingredients in conventional manner to form a compacted mass in multiple layers, which comprises the core of the dosage form (also referred to herein as “tablet core”). The tablet core may be prepared using conventional tablet excipients and formulation compression methods. Thus, the core typically comprises the active agent or agents along with excipients that impart satisfactory processing and compression characteristics such as diluents, binders and lubricants. Additional excipients that may form part of the core of the device include disintegrants, flavourants, colorants, release modifying agents and/or solubilising agents such as surfactants, pH modifiers and complexation vehicles. Typically, the active agent and excipients are thoroughly mixed prior to compression into a solid core. The core of the device may be formed by wet granulation methods, dry granulation methods or by direct compression. The core may be produced according to any desired pre-selected shape such as bi-convex, hemi-spherical, near hemi-spherical, round, oval, generally ellipsoidal, oblong, generally cylindrical or polyhedral, e.g. a triangular prism shape. The term “near hemi-spherical” is intended to be construed in the manner described in U.S. Pat. No. 5,004,614. Suitably, the core is formulated into a bi-convex shape, e.g. having two domed opposite surfaces.

The core may be coated with a suitable pH dependent erodable material by any pharmaceutically acceptable coating method. Examples include coating methods disclosed in U.S. Pat. No. 5,004,614 and film coating, sugar coating, spray coating, dip coating, compression coating, electrostatic coating. Typical methods include spraying the coating onto the tablet core in a rotating pan coater or in a fluidised bed coater until the desired coating thickness is achieved. Suitably the coating is provided to add about 4 to 8 mg/cm² or 5-7 mg/cm² of dry polymer around the tablet surface area. Typically this results in an increase in weight (relative to the core) of from 3-10% or 5-10% by weight. Suitably, the coating has a thickness in the range 0.05 to 0.5 mm.

As indicated above, the oral dosage form of the present invention is considered to be suitable for once daily administration and during use is indicated to provide a therapeutic effect over an extended period of time, such as up to 24 hours, for example, up to 12, 14, 16, 18, 20 and 24 hours, per unit dose.

As used herein, the term “modified release” means a composition which has been designed to produce a desired pharmacokinetic profile by choice of formulation. Modified release also includes modified release compositions in combination with non-modified release compositions. For example, the term “modified release” shall comprise delayed, pulsed and sustained release either alone or in any combination.

In one aspect the modified release composition provides delayed release of a pharmaceutically acceptable weak base, such as Compound A or a pharmaceutically acceptable salt or solvate thereof. Delayed release is conveniently obtained by use of a gastric resistant formulation such as an enteric formulation. Such an enteric formulation may comprise multi-particulates, such as multi-particulate spheres, coated with a gastric resistant polymer. Suitable, gastric resistant polymers include polymers derived from methacrylates, cellulose acetate phthalate, polyvinyl acetate phthalate and hydroxypropyl methylcellulose phtahlate. Examples of such polymers include Eudragit L100-55™ (Poly(methacrylic acid, ethyl acrylate) 1:1) for example as Eudragit L30D-55™ or Eudragit FS 30D™, Aquateric™ (cellulose acetate phthalate), Sureteric™ (polyvinyl acetate phthalate), HPMCP-HP-55S™ (hydroxypropyl methylcellulose phtahlate).

The multiparticulates include coated drug-coated non-pareil substrates, such as lactose spheres, or drug containing non-pareil substrates, such as drug containing lactose spheres. Such multiparticulates are coated as required with an appropriate enteric formulation, for example a polymethacrylate polymer. An example of a suitable polymethacrylate polymer is Eudragit L100-55™ (Poly(methacrylic acid, ethyl acrylate) 1:1), for example as Eudragit L30D-55™ or Eudragit FS 30D™.

In a further aspect the modified release composition provides sustained release of a pharmaceutically acceptable weak base, such as Compound A or a pharmaceutically acceptable salt or solvate thereof, for example providing release of the active agent over a time period of up to 26 hours; suitably in the range of 4 to 24 hours; preferably in the range of 12 to 24 hours.

Sustained release is typically provided by use of a sustained release matrix, usually in tablet form, such as disintegrating, non-disintegrating or eroding matrices.

Sustained release is suitably obtained by use of a non-disintegrating matrix tablet formulation. Suitable non disintegrating matrix tablet formulations are provided by the incorporation of methacrylates, cellulose acetates, carbomers and hydroxypropyl methylcellulose phtahlate into the tablet. Examples of suitable materials include Eudragit RS™ (Poly(ethyl acrylate, methyl methacrylate, trimethylammonioethyl methacrylate chloride) 1:2:0.1), Eudragit RL™ (Poly(ethyl acrylate, methyl methacrylate, trimethylammonioethyl methacrylate chloride) 1:2:0.2), Carbopol 971 P™ (carbomer), HPMCP-HP-55S™ (hydroxypropyl methylcellulose phtahlate).

Sustained release is further obtained by use of a disintegrating matrix tablet formulation, for example by incorporating methacrylates, methylcellulose or hydroxypropyl methylcellulose into the tablet. Examples of suitable materials include Eudragit L™ (Poly(methacrylic acid, ethyl acrylate) 1:1) and Methocel K4M™ (hydroxypropyl methylcellulose).

Sustained release can also be achieved by using multiparticulates coated with semipermeable membranes. The multiparticulates include coated drug-coated non-pareil substrates, such as lactose spheres, or drug containing substrates, such as drug containing lactose/Avicel™ (microcrystalline cellulose) spheres. Such multiparticulates are coated as required with the appropriate semi-permeable membranes, such as ethylcellulose polymer.

In yet a further aspect the modified release composition provides pulsed release of a pharmaceutically acceptable weak base, such as Compound A or a pharmaceutically acceptable salt or solvate thereof, for example providing up to 4, for example 2, pulses of active agent per 24 hours.

Suitable materials for an immediate release composition, such as the first composition, include saccharoses, for example lactose and maltose. Most suitably, the immediate release composition is predominantly lactose. More suitably, the immediate release composition consists essentially of lactose and magnesium stearate.

The quantity of the pharmaceutically active weak base or a pharmaceutically acceptable salt or solvate thereof to be used in accordance with the present invention is a matter to be determined based upon typical pharmaceutical considerations, e.g. known dosages for the pharmaceutically active weak base or a pharmaceutically acceptable salt or solvate thereof, and is not limited by the process of this invention.

In particular, where Compound A or a pharmaceutically salt or solvate thereof is used in accordance with the present invention, a suitable dosage range is up to 12 mg, for example, 1 to 12 mg. Thus, suitable dosage forms comprise 1, 2, 3, 4, 5, 6, 7, 8, 9, 10, 11 or 12 mg of Compound A or a pharmaceutically acceptable salt or solvate thereof.

Particular dosage forms comprise 2 to 4 mg of Compound A or a pharmaceutically acceptable salt or solvate thereof.

Particular dosage forms comprise 4 to 8 mg of Compound A or a pharmaceutically acceptable salt or solvate thereof.

Particular dosage forms comprise 8 to 12 mg of Compound A or a pharmaceutically acceptable salt or solvate thereof.

One dosage form comprises 1 mg of Compound A or a pharmaceutically acceptable salt or solvate thereof.

One dosage form comprises 2 mg of Compound A or a pharmaceutically acceptable salt or solvate thereof.

Preferred dosage forms comprise 4 mg of Compound A or a pharmaceutically acceptable salt or solvate thereof.

Preferred dosage forms comprise 8 mg of Compound A or a pharmaceutically acceptable salt or solvate thereof.

The amount of Compound A or a pharmaceutically acceptable salt or solvate thereof present in the first composition and the second composition may be varied in accordance with the desired dissolution profile.

For example, where the oral dosage form comprises 8 mg of Compound A or a pharmaceutically acceptable salt or solvate thereof, the tablet core suitably comprises a layer comprising 1 mg of Compound A or a pharmaceutically salt or solvate thereof, and a layer comprising 7 mg of Compound A or a pharmaceutically salt or solvate thereof. Alternatively, the tablet core may comprise a layer comprising 4 mg of Compound A or a pharmaceutically salt or solvate thereof, and a layer comprising 4 mg of Compound A or a pharmaceutically salt or solvate thereof. More suitably, the tablet core comprises a layer comprising 2 mg of Compound A or a pharmaceutically salt or solvate thereof, and a layer comprising 6 mg of Compound A or a pharmaceutically salt or solvate thereof. Preferably, the tablet core comprises a layer comprising 3 mg of Compound A or a pharmaceutically salt or solvate thereof, and a layer comprising 5 mg of Compound A or a pharmaceutically salt or solvate thereof.

Where the oral dosage form comprises 2 mg of Compound A or a pharmaceutically acceptable salt or solvate thereof, the tablet core suitably comprises a layer comprising 0.75 mg of Compound A or a pharmaceutically salt or solvate thereof, and a layer comprising 1.25 mg of Compound A or a pharmaceutically salt or solvate thereof.

Where the oral dosage form comprises 4 mg of Compound A or a pharmaceutically acceptable salt or solvate thereof, the tablet core suitably comprises a layer comprising 1.5 mg of Compound A or a pharmaceutically salt or solvate thereof, and a layer comprising 2.5 mg of Compound A or a pharmaceutically salt or solvate thereof.

By adjustment of the release rates of the first and second compositions, and adjusting the other variables mentioned above and the surface area of the exposed core, the release rates in the different environmental conditions can be harmonised to obtain comparable release rates under different body environments, and so achieve more constant dosing to a patient.

Preferably the dissolution rates of the oral dosage forms of this invention are arranged, for example by routine adjustment of the erodable coating and dimensions of the opening(s), so that the rate of release is substantially similar in the different pH environments experienced by the dosage form on administration. Dissolution rates may be assessed by in vitro testing in solutions of the appropriate pHs. For example, when comparing dissolution in the stomach and intestine, tests may be carried out initially at pH 1.5 with a transfer to pH 6.8 after 2 hours or 4 hours, as an assumed time for residence in the stomach before emptying into the intestines of a notional patient in respectively fasted and fed conditions.

As mentioned above, Compound A or a pharmaceutically acceptable salt or solvate thereof when administered in an oral dosage form of this invention is indicated to be useful for the treatment and/or prophylaxis of diabetes mellitus, conditions associated with diabetes mellitus and certain complications thereof; Alzheimer's Disease, psoriasis, asthma, atherosclerosis, metabolic syndrome, impaired glucose tolerance and impaired fasting glucose (hereinafter referred to as the ‘Disorders of the Invention’). Suitably, Compound A or a pharmaceutically acceptable salt or solvate thereof when administered in an oral dosage form of this invention is indicated to be useful in the treatment and/or prophylaxis of diabetes mellitus, conditions associated with diabetes mellitus and certain complications thereof. Suitably, Compound A or a pharmaceutically acceptable salt or solvate thereof when administered in an oral dosage form of this invention is indicated to be useful in the treatment and/or prophylaxis of Alzheimers Disease. Suitably, Compound A or a pharmaceutically acceptable salt or solvate thereof when administered in an oral dosage form of this invention is indicated to be useful in the treatment and/or prophylaxis of psoriasis. Suitably, Compound A or a pharmaceutically acceptable salt or solvate thereof is indicated to be useful in the treatment and/or prophylaxis of asthma. Suitably, Compound A or a pharmaceutically acceptable salt or solvate thereof when administered in an oral dosage form of this invention is indicated to be useful in the treatment and/or prophylaxis of atherosclerosis. Suitably, Compound A or a pharmaceutically acceptable salt or solvate thereof when administered in an oral dosage form of this invention is indicated to be useful in the treatment and/or prophylaxis of metabolic syndrome. Suitably, Compound A or a pharmaceutically acceptable salt or solvate thereof is indicated to be useful in the treatment and/or prophylaxis of impaired glucose tolerance. Suitably, Compound A or a pharmaceutically acceptable salt or solvate thereof when administered in an oral dosage form of this invention is indicated to be useful in the treatment and/or prophylaxis of impaired fasting glucose.

In a preferred embodiment the present invention provides a method for the treatment and/or prophylaxis of the Disorders of the Invention which method comprises administering an oral dosage form of this invention comprising Compound A or a pharmaceutically acceptable salt or solvate thereof, to a human or non-human mammal in need thereof.

In a further preferred embodiment the present invention provides an oral dosage form of the invention comprising Compound A or a pharmaceutically acceptable salt or solvate thereof for use in the treatment and/or prophylaxis of the Disorders of the Invention.

As used herein, the term “pharmaceutically acceptable” embraces compounds, compositions and ingredients for both human and veterinary use. For example the term “pharmaceutically acceptable salt” embraces a veterinarily acceptable salt. In particular, suitable pharmaceutically acceptable salted forms of Compound A include those described in European Patent Number 0 306 228 and International Patent Application, Publication Number WO 94/05659. A particularly preferred form of Compound A is the maleate salt.

Suitable pharmaceutically acceptable solvates include hydrates.

As used herein, the term “Cₘₐₓ” shall mean the mean maximum plasma level concentration.

As used herein the term “AUC” shall mean the mean area under the plasma concentration versus time curve over the dosing interval at steady state.

No adverse toxicological effects are indicated in the above mentioned treatments.

All publications, including but not limited to patents and patent applications, cited in this specification are herein incorporated by reference as if each individual publication were specifically and individually indicated to be incorporated by reference herein as though fully set forth.

In the following Examples, tablet cores were formed by conventional means by mixing together the active ingredients with excipients and compressing to form multilayer tablet core. These Examples are intended to be by way of illustration rather than limitation of the present invention, and Compound A is used simply as one example of a weak base suitable for use with the present invention.

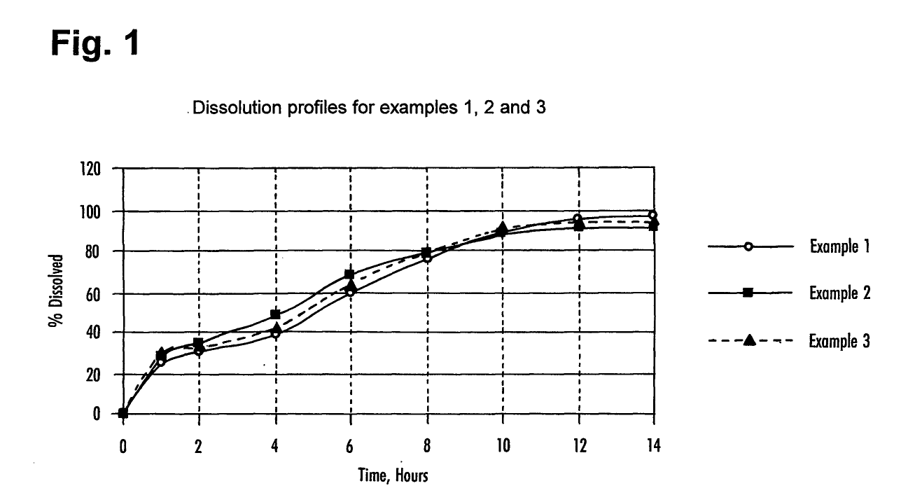

FIG. 1 is a graph of dissolution against time for formulations of oral dosage form in accordance with Examples 1, 2 and 3 of this application.

### EXAMPLE 1

A core was formed from the following compositions:

### %w/w

First Composition

Immediate Release Layer

Compound A (as maleate salt) 5.3
Lactose 94.17
Yellow iron oxide 0.03
Magnesium stearate 0.5
   Second Composition
   Modified Release Layer
Compound A (as maleate salt) 5.3
HPMC 30.0
Lactose 62.7
Colloidal silicon dioxide 0.5
Magnesium stearate 1.5
   by compression to form 7 mm normal concave bilayer tablets of 200 mg (50 mg of the immediate release layer and 150 mg of the modified release layer).

The tablet cores were coated with a HPMC-based sub-coat and a polymethacrylate resin soluble at pH 5.5 to a total weight of 217.3 mg.

An opening of diameter 3.0 mm was drilled through the coating in each of the two primary surfaces of the coated cores to expose the surface of the core.

### EXAMPLE 2

A core was formed from the following compositions:

### %w/w

First Composition

Immediate Release Layer

Compound A (as maleate salt) 5.3
Lactose 94.17
Yellow iron oxide 0.03
Magnesium stearate 0.5
   Second Composition
   Modified Release Layer
Compound A (as maleate salt) 5.3
HPMC 30.0
Lactose 62.7
Colloidal silicon dioxide 0.5
Magnesium stearate 1.5
   by compression to form 7 mm normal concave bilayer tablets of 200 mg (50 mg of the immediate release layer and 150 mg of the modified release layer).

The tablet cores were coated with a HPMC-based sub-coat and a polymethacrylate resin soluble at pH 5.5 to a total weight of 217.3 mg.

An opening of diameter 4.0 mm was drilled through the coating in each of the two primary surfaces of the coated cores to expose the surface of the core.

### EXAMPLE 3

A core was formed from the following compositions:

### %w/w

First Composition

Immediate Release Layer

Compound A (as maleate salt) 7.95
Lactose 91.52
Yellow iron oxide 0.03
Magnesium stearate 0.5
   Second Composition
   Modified Release Layer
Compound A (as maleate salt) 4.4
HPMC 30.0
Lactose 63.6
Colloidal silicon dioxide 0.5
Magnesium stearate 1.5
   by compression to form 7 mm normal concave bilayer tablets of 200 mg (50 mg of the immediate release layer and 150 mg of the modified release layer).

The tablet cores were coated with a HPMC-based sub-coat and a polymethacrylate resin soluble at pH 5.5 to a total weight of 217.3 mg.

An opening of diameter 3.0 mm was drilled through the coating in each of the two primary surfaces of the coated cores to expose the surface of the core.

Dissolution profiles for the dosage forms of Examples 1, 2 and 3 are shown in FIG. 1 of the accompanying drawings.

A Study to Evaluate the Pharmacokinetics of Six Modified Release Formulations of Compound A (8 mg) in Healthy Volunteers After Repeat Dosing

Primary Objective

To compare the repeat dose pharmacokinetics of six modified release formulations of Compound A to those of an immediate release formulation of Compound A in the fasted state.

Secondary Objectives

To investigate the effect of food on the repeat dose pharmacokinetics of six modified release formulations of Compound A.

To compare the single dose pharmacokinetics of six modified release formulations of Compound A to those of an immediate release formulation of Compound A in the fasted state.

To investigate the pharmacokinetics of six modified release formulations and an immediate release formulation of Compound A under repeat dosing relative to single dosing.

To assess the tolerability of repeat oral doses of each of the six modified release formulations of Compound A.

Study Design

This was a randomized, open-label, three-period, crossover study with three parallel groups conducted in healthy volunteers. Each subject participated in three study sessions separated by a washout period of at least 48 hours. In each study session, subjects were randomized to receive either the immediate release formulation of Compound A (4 mg) twice daily, or two of six modified release formulations of Compound A (8 mg). The immediate release formulation of Compound A (4 mg) was given twice a day for six days and in the morning of the seventh day under fasted conditions. Each modified release formulation of Compound A (8 mg) was given once a day for seven days, under fasted conditions, then under fed conditions on the eighth day.

Number and Nature of Subjects

A sufficient number of subjects were enrolled so that 42 evaluable subjects completed the study (i.e. at least 13 subjects per parallel group). Subjects were healthy adult male and female volunteers between 18 and 65 years of age (inclusive).

Treatment Administration

During each study session, subjects in each of three parallel groups received either repeat oral doses of immediate release formulation of Compound A (4 mg) twice a day for seven days, or two of six modified release formulations of Compound A (8 mg) administered once a day for eight days.

Criteria for Evaluation

Pharmacokinetics (AUC and Cmax) for immediate release and modified release formulations were the primary pharmacokinetic parameters. The secondary pharmacokinetic parameters were tₘₐₓ and t_{1/2} of immediate release and modified release formulations. Plasma specimens for Compound A pharmacokinetic analysis were obtained prior to study medication administration every day in each study session; over a 12-hour interval on Days one and seven when dosed with the immediate release formulation of Compound A (Regimen A); and during a 24-hour interval on Days one, seven and eight when dosed with a modified release formulation of Compound A (Regimens B-G). Plasma concentrations for pharmacokinetic analysis of Compound A were determined by validated assay methodologies.

Safety Results

There were no deaths or serious adverse events reported during the study.

Pharmacokinetic Results

Of the six Compound A modified release (MR) formulations (IR and MR components) tested, one formulation (Regimen F) was selected to carry forward into the confirmatory clinical study. The PK results for this modified release formulation in the fasted and-fed state are provided in Table 1 below.

TABLE 1
Point Estimates and Confidence Intervals for AUC (ng · h/mL),
Cmax (ng/mL), tmax (h) and t_{1/2} (h) of Compound A modified release
formulation Compared to Compound A immediate release Formulation
on Day 1 and 7 in Fasted State, and Compound A modified release
Formulation on Day 8 in Fed State

| | | | |
|---|---|---|---|
| | | Point | |
| Parameter | Comparison | Estimate | 90% CI |

Compound A MR vs IR on Day 1

| | | | |
|---|---|---|---|
| AUC(0-∞) | MR (day 1):IR (day 1) | 1.00 | (0.92, 1.09) |
| (ng · h/mL)¹ | | | |
| Cmax (ng/mL)¹ | MR (day 1):IR (day 1) | 0.88 | (0.77, 1.01) |
| tmax (hr)² | MR (day 1):IR (day 1) | 3.00 | (2.75, 3.25) |
| t½ (hr)¹ | MR (day 1):IR (day 1) | 2.19 | (1.87, 2.58) |

Compound A vs IR on Day 7

| | | | |
|---|---|---|---|
| AUC(0-24) | MR (day 7):IR (day 7) | 0.99 | (0.92, 1.08) |
| (ng · h/mL)¹ | | | |
| Cmax (ng/mL)¹ | MR (day 7):IR (day 7) | 0.94 | (0.82, 1.08) |
| tmax (hr)² | MR (day 7):IR (day 7) | 3.25 | (3.00, 4.25) |
| t½ (hr)¹ | MR (day 7):IR (day 7) | 1.92 | (1.63, 2.27) |

Compound A MR Fed vs MR Fasted

| | | | |
|---|---|---|---|
| AUC(0-24) | MR (day 8):MR (day 7) | 0.98 | (0.90, 1.06) |
| (ng · h/mL)¹ | | | |
| Cmax (ng/mL)¹ | MR (day 8):MR (day 7) | 1.04 | (0.89, 1.21) |
| tmax (hr)² | MR (day 8):MR (day 7) | 4.01 | (3.00, 5.50) |
| t½ (hr)¹ | MR (day 8):MR (day 7) | 0.68 | (0.57, 0.82) |

¹represents the ratio of adjusted geometric means.
²represents the estimated median difference.

Compared to the Compound A (4 mg) twice daily, immediate release reference formulation, the 90% Cl of the geometric mean ratio for AUC of the Compound A modified release formulation was within 80-125% following single (Day 1) and repeat dose (Day 7), and for Cmax following repeat dose (Day 7). Furthermore, the 90% Cl was within 80-125% for AUC and Cmax when the MR formulation was administered with a high fat meal (Day 8) compared to the fasted state (Day 7). None of the other modified release formulations were bioequivalent to the immediate release formulation for both AUC and Cmax, as well as no relevant effect of food.

A pharmacokinetic/pharmacodynamic (PK/PD) model was previously developed to describe the delayed and indirect effects of Compound A on fasting plasma glucose and hemoglobin A1c concentrations in diabetic patients (May 25, 2000 FDA Briefing Document submission to Avandia IND 43,468, Serial No. 266). The model estimated the concentration of Compound A associated with half-maximal stimulation (SC50) of glucose utilization to be ˜52ng/ml. The time above SC50 was determined for the Compound A 4 mg twice daily, immediate release and 8 mg modified release formulation and is shown in Table 2 below.

TABLE 2
Mean (SD) of Time (hrs) Above SC50 (˜52 ng/ml) for
Compound A 4 mg IR and Compound A 8 mg MR on Days 1
and 7, and Compound A 8 mg MR on Day 8 (Fed State)

| | Day 1 | Day 7 | Day 8 (Fed) |
|---|---|---|---|
| 4 mg BID IR | 15.7 (2.6) | 16.3 (3.3) | |
| 8 mg MR | 15.5 (4.2) | 15.4 (3.3) | 16.0 (3.5) |

On single and repeat dosing, the time above SC50 was consistent between the 4 mg IR administered twice daily and the 8 mg MR Compound A formulation administered once daily. Furthermore, the presence of food appeared to have little impact on the time above SC50.

### CONCLUSION

A once-a-day Compound A (8 mg) modified release tablet formulation (comprising an IR and MR component) has been identified that is bioequivalent to twice daily Compound A (4 mg) immediate release formulation during repeat dosing. After administration with a high-fat breakfast, the AUC and Cmax of the Compound A modified release formulation at steady-state was equivalent to those observed under fasting conditions.

Furthermore, the time above SC50 was consistent between the 4 mg immediate release formulation administered twice daily and the 8 mg modified release formulation administered once daily. The presence of food appeared to have little impact on the time above SC50.

Multiple doses of Compound A 4 mg immediate release formulation or modified release formulations of Compound A (8 mg) administered under fasting conditions were generally safe and well-tolerated by healthy subjects.

1-21. (canceled)

22. An oral dosage form comprising a first composition and a second composition, wherein each composition comprises a drug compound, which is 5-[4-[2-(N-methyl-N-(2pyridyl)amino)ethoxy]benzyl]thiazolidine-2,4-dione or a pharmaceutically acceptable salt or solvate thereof, and a pharmaceutically acceptable carrier therefor,
wherein the first and second compositions are arranged to release the drug compound at differing release rates on administration such that the rate of release of the drug compound from the dosage form is substantially independent of pH.

23. An oral dosage form according to claim 22, wherein the release rate of the drug compound from the first composition is substantially greater than the release rate of the drug compound from the second composition.

24. An oral dosage form according to claim 22, wherein the first composition is an immediate release composition.

25. An oral dosage form according to claim 22, wherein the second composition is a modified release composition.

26. An oral dosage form according to claim 22, wherein the rate of release of the drug compound from at least one of the first composition and the second composition is a modified release.

27. An oral dosage form according to claim 26, comprising a third composition comprising substantially no drug substance.

28. An oral dosage form according to claim 27, wherein said third composition is an enteric composition.

29. An oral dosage form according to claim 28, wherein said third composition comprises one or more openings extending substantially completely through the third composition, thereby exposing at least one surface of
(a) the first composition,
(b) the second composition, or
(c) the first and second compositions,
to the environment of use.

30. An oral dosage form according to claim 22, wherein the first composition is arranged to release substantially all of the drug compound in the stomach.

31. An oral dosage form according to claim 22, wherein the second composition is arranged to release substantially all of the drug compound in the small intestine.

32. An oral dosage form according to claim 22, which dosage form is arranged to release the drug compound such that the mean maximum plasma level concentration value of the drug is maintained substantially independent of food during use.

33. An oral dosage form according to claim 22, which dosage form is arranged to release the drug compound such that the mean area under the plasma concentration versus time curve over the dosing interval at steady state is maintained substantially independent of food during use.

34. An oral dosage form according to claim 22, which dosage form is arranged to release the drug compound so that both the mean maximum plasma level concentration and the mean area under the plasma concentration versus time curve over the dosing interval at steady state are maintained substantially independent of food during use.

35. An oral dosage form according to claim 22, comprising,
(i) an erodable core, which core comprises the first composition and the second composition; and
(ii) an erodable coating around said core, which coating comprises one or more openings extending substantially completely through said coating but not penetrating said core and communicating from the environment of use to said core,
wherein release of the drug compound from the erodable core occurs substantially through the one or more openings and through erosion of said erodable coating under pre-determined pH conditions.

36. An oral dosage form according to claim 35, wherein the first composition is formulated to provide an immediate release of the drug compound on contact with aqueous media.

37. An oral dosage form according to claim 35, wherein the second composition is formulated to provide a modified release of the drug compound on contact with aqueous media.

38. An oral dosage form according to claim 22, wherein the dosage form is a tablet form.

39. A process for preparing the oral dosage form according to claim 22, which process comprises at least the steps of:
(i) formulating the drug compound into the first composition; and
(ii) formulating the drug compound into the second composition;
whereby the first and second compositions are formulated to release drug at differing release rates on administration such that the rate of release of the drug compound from the dosage form is substantially independent of pH.

40. A process for the preparation of the oral dosage form according to claim 35, which process comprises:
(a) formulating the erodable core comprising the drug compound and a pharmaceutically acceptable carrier therefor;
(b) coating the core with the erodable coating; and
(c) forming one or more openings in the coating, said openings extending substantially completely through said coating but not penetrating said core and communicating from the environment of use to said core.

41. A method for the treatment of a diabetes mellitus, Alzheimer's Disease, psoriasis, asthma, atherosclerosis, metabolic syndrome, impaired glucose tolerance or impaired fasting glucose in a human or non-human mammal, which method comprises administering the oral dosage form according to claim 22 to said human or non-human mammal.
